(19) United States
(12) Patent Application Publication
(10) Pub. No.: US 2019/0344057 A1
(43) Pub. Date: Nov. 14, 2019

(54) NANOFLUIDIC PERISTALTIC PUMPS AND METHODS OF USE

(71) Applicant: Massachusetts Institute of Technology, Cambridge, MA (US)

(72) Inventors: Michael J. Cima, Winchester, MA (US); Robert Langer, Newton, MA (US); Ritu Raman, Cambridge, MA (US)

(21) Appl. No.: 16/411,907

(22) Filed: May 14, 2019

Related U.S. Application Data
(60) Provisional application No. 62/671,020, filed on May 14, 2018.

(51) Int. Cl. A61M 27/00; A61M 31/00; A61M 5/142; F16K 99/00; F04B 43/04

(52) U.S. Cl. CPC A61M 27/006; A61M 31/002; A61M 5/14276; F16K 2099/0088; F04B 43/043; A61M 2205/3331; B01L 2400/0661; F16K 99/0038

(57) ABSTRACT

A nanofluidic peristaltic pump includes an elongated tubular member having a first end, an opposed second end, and an elastic wall defining a flow channel extending between the first and second ends; and a series of shape memory alloy actuator wires extending across and at least partially around the outer surface of the elastic wall at spaced positions along the length of the tubular member, wherein the actuator wires are configured to reversibly and directly compress the wall, and thereby constrict regions of the flow channel, upon an electrothermally induced phase transition of the shape memory alloy. With the flow channel at the first end of the tubular member in fluid communication with a fluid source, an electric current is delivered to the actuator wires to sequentially activate and deactivate them and cause fluid to flow through the flow channel from the first end toward the second end.

### CROSS-REFERENCE TO RELATED APPLICATIONS

This application claims priority benefit to U.S. Provisional Patent Application No. 62/671,020, filed May 14, 2018, which is incorporated herein by reference.

### STATEMENT REGARDING FEDERALLY SPONSORED RESEARCH

This invention was made with Government support under Grant No. R01 EB016101 awarded by the National Institutes of Health. The Government has certain rights in the invention.

### BACKGROUND

The present disclosure is generally in the field of pumps, and more particularly microfluidic and nanofluidic pumps, including, but not limited to, such pumps configured for biomedical applications such as those for implantation in the body of a patient for delivery and/or withdrawal of fluids.

There is a significant and growing need for targeted patient-specific therapies for disease. These therapies often require mechanisms for identifying individual patient pathologies, as well as delivering precise doses of drugs to target sites within the body of patient. Fluid pumps are often used in these methods, and for chronic use it would be desirable to be able to implant the pump into the patient, for example subcutaneously. However, conventional pumps have various drawbacks, rendering them unsuitable or undesirable for implantation and/or unable to provide precise low-volume (nL/s) fluid control.

For example, some pumps are unidirectional and rely on syringe-based designs, as in U.S. Pat. No. 6,375,638, or roller-based designs, as in U.S. Pat. Nos. 6,743,204 and 6,733,476, and have not been reported to produce single stroke volumes of less than 200 nL [Au, et al., Micromachines, 2(2), pp. 179-220 (2011); Cima, Ann. Rev. Chem. & Biomolec. Eng., 2, pp. 355-78 (2011); Fong, et al., Lab on a Chip, 15(4), pp. 1050-58 (2015)]. In some designs, a shape memory alloy actuator is used to drive a separate force applying member, which in turn acts upon a force receiving member, resulting in pumping. It would be desirable to reduce the number and/or size of the parts required to produce the movement of fluid through the pump, and it would be desirable to be able precisely deliver smaller fluid volumes.

Bidirectional implantable pumps are known, but have limited utility due to their large flow rates (μL/s) and their use of magnetically-susceptible materials, which renders them MRI-incompatible [Ludvig, et al., J. Neuroscience Methods, 203(2), pp. 275-83 (2012)]. This reduces their suitability for applications that require chronic implantation, and for applications that require more precise low-volume (nL/s) fluid control, such as neural implants.

It would be desirable to provide a pump suitable for long-term implantation in a patient to deliver very precise and very small quantities of a drug into a targeted site in a patient's brain via a needle connected to the pump, and/or to withdraw very small quantities of cerebrospinal fluid, for example for diagnostic analysis. In such a system, it would be desirable to make the overall size of the pump very small, e.g., having a narrow profile, and to keep the actuation parts of the pump as static as possible to avoid movement of the needle. Conventional pumps, however, are too large, are limited to large flow rates, are incompatible with MRI, and/or provide only unidirectional flow.

In sum, it would be desirable to provide new, improved nanofluidic pumps that overcomes one or more of the foregoing disadvantages and deficiencies.

### SUMMARY

In one aspect, a nanofluidic peristaltic pump is provided. In some embodiments, the pump includes: an elongated tubular member having a first end, an opposed second end, and a wall defining a flow channel extending between the first and second ends; and a series of actuator wires, each comprising a shape memory alloy, wherein the actuator wires extend across and at least partially around the outer surface of the elastic wall at spaced positions along the length of the tubular member, the actuator wires being configured to reversibly and directly compress the wall, and thereby constrict regions of the flow channel, upon an electrothermally induced phase transition of the shape memory alloy. The pump may include one or more check valves for mitigating or eliminating backflow. The pump may provide bidirectional flow.

In another aspect, a method of pumping a fluid is provided. In some embodiments, the method includes: providing one of the disclosed nanofluidic peristaltic pumps with the flow channel at the first end of the tubular member in fluid communication with a fluid source; and delivering an electric current to at least first portion of the actuator wires to sequentially activate and deactivate them and cause the fluid to flow through the flow channel from the first end toward the second end. The step of providing the nanofluidic peristaltic pump may include implanting or inserting the nanofluidic peristaltic pump into the body of a patient.

In still another aspect, a method is provided for delivering a drug into a patient and/or for withdrawing a sample of a biological fluid. The method may include providing one of the disclosed nanofluidic peristaltic pumps and subcutaneously implanting the pump in the patient.

Other features and aspects of the disclosure will be apparent or will become apparent to one skilled in the art upon examination of the following figures and the detailed description.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments.

Elements and/or components in the figures are not necessarily drawn to scale.

FIG. 1 is a perspective view of one embodiment of a nanofluidic peristaltic pump in accordance with the present invention.

FIG. 2 is a perspective view of another embodiment of a nanofluidic peristaltic pump in accordance with the present invention.

FIGS. 3A-3B are cross-sectional views of yet another embodiment of a nanofluidic peristaltic pump in accordance with the present invention.

FIG. 4 is a diagram of one embodiment of a system including a fluid source and a nanofluidic peristaltic pump in accordance with the present invention.

### DETAILED DESCRIPTION

Improved nanofluidic peristaltic pumps and methods of operating the pumps and transporting incompressible fluids have been developed. In some particular embodiments, the nanofluidic peristaltic pump is designed to control bidirectional fluid flow with nanoliter precision. In some particular embodiments, the pump has a slim profile, enabling minimally invasive insertion (e.g., subcutaneous implantation) in a patient's body and ready interfacing with implanted medical devices. The pump can be used to precisely deliver drugs to, or sample fluids from, the body through these interfaces.

Conventional pumps that include a shape memory alloy require a force-applying member which in turn acts upon a force-receiving member to cause pumping. In contrast, the presently disclosed pumps beneficially omit such additional force-applying members. That is, the presently disclosed pumps do not need and do not include pistons, rollers, or other force-applying members in addition to the shape memory alloy components. The newly developed nanofluidic peristaltic pump design advantageously uses shape memory alloy wires as both the actuator and the force applying member: The contraction of the shape memory alloy wire directly compresses the force receiving member, which is a compliant fluidic channel or tube. The simpler design beneficially requires fewer moving parts, which streamlines the design and enables ready miniaturization.

The design also advantageously enables a pump having a single stroke volume of less than 200 nL. In some embodiments, the pump is configured to produce a single stroke volume between about 100 nL and 200 nL. In some other embodiments, the pump is configured to produce a single stroke volume between about 10 nL and 100 nL. In some other embodiments, the pump is configured to produce a single stroke volume between about 1 nL and 10 nL.

It is noted that the pump beneficially is able to pump a liquid even when the flow channel is not completely filled with liquid. For example, the pump is still operable when the flow channel is partially filled with air.

Reference to “about” a value or parameter herein includes (and describes) embodiments that are directed to that value or parameter per se. For example, “about 100 nL” includes 100 nL. The term “about” indicates the value of a given quantity can include quantities ranging within 10% of the stated value.

The term “patient” as used herein refers to a mammal, including humans. A patient includes, but is not limited to, human, bovine, equine, feline, canine, rodent, or primate. In some embodiments, the patient is human.

### The Pump

In one aspect, the nanofluidic peristaltic pumps, which may be bidirectional pumps, include an elongated tubular member having a first end, an opposed second end, and a wall (e.g., an elastic wall) defining a flow channel extending between the first and second ends; and a series of actuator wires, each comprising a shape memory alloy, wherein the actuator wires extend across and at least partially around the outer surface of the elastic wall at spaced positions along the length of the tubular member. That is, the actuator wires are in contact with the wall at positions spaced from one another. The actuator wires are configured to reversibly and directly compress the elastic wall, and thereby constrict regions of the flow channel, upon an electrothermally induced phase transition of the shape memory alloy. The reversibility may be complete or partial so long as the pumping functionality is provided.

Contraction of the wire length results in displacement of fluid within the tube. The magnitude, sequence, and frequency of wire contraction can be used to control the rate and direction of fluid flow. Sequential contraction of the wire actuators can drive directional fluid flow, with the sequence of contraction determining the direction and rate of fluid flow. The flow rate can be tuned further by regulating the number of wires, the degree of wire pre-tension in its passive unpowered state, the degree of wire contraction (controlled by amplitude of current flow) in its active state, the duration of wire contraction, and the duration of overlap between sequential contracting wires.

In some embodiments, a bidirectional nanofluidic peristaltic pump is provided. In one embodiment, the pump includes (i) an elongated, elastomeric tubular member having a first end, an opposed second end, and a wall defining a flow channel extending between the first and second ends; (ii) a series of shape memory alloy (e.g., nitinol) actuator wires extending around at least part of the outer surface of the wall of the elastomeric tubular member, the actuator wires being in contact with the wall at positions spaced from one another; and (iii) a power source and controller operably connected to the series of actuator wires and configured to selectively sequentially deliver an electric current to each of the actuator wires to electrothermally induce a phase transition of the shape memory alloy, wherein the actuator wires, upon the electrothermally induced phase transition of the shape memory alloy, are configured to reversibly and directly compress the wall, and thereby constrict regions of the flow channel. The elastomeric tubular member may be formed of silicone or polyurethane, for example. In some embodiments, the pump has a series of from 3 to 300 actuator wires. In some embodiments, the pump has from 3 to 30 actuator wires. For example, the pump may include from 3 to 10 actuator wires, e.g., 3, 4, 5, 6, 7, 8, 9, or 10 actuator wires.

Some of these actuator wires may also be configured to operate as check valves.

In some embodiments, the pump includes a substrate on which the elastomeric tubular member is disposed and to which the actuator wires are affixed. For example, the substrate may be a rigid base supporting the elastomeric tubular member and actuator wires.

In some embodiments, each of the actuator wires has a diameter from about 25 μm to about 100 μm. For example, the wire diameter may be 50 μm, 60 μm, 70 μm, 80 μm, 90 μm, or within a range bound by a pair of these values.

In some embodiments, the flow channel has a diameter from about 20 μm to about 1000 μm. For example, the flow channel diameter may be 20 μm, 30 μm, 40 μm, 50 μm, 60 ram, 70 rpm, 80 μm, 90 μm, 100 μm, 150 μm, 200 μm, 500 μm, or within a range bound by a pair of these values.

In some embodiments, each of the actuator wires has a diameter from about 50 μm to about 100 μm, and the flow channel has a diameter from about 20 μm to about 1000 am. For example, the actuator wire diameter may be about 50 μm, and the flow channel diameter may be from 50 μm to 150 μm, e.g., about 100 am.

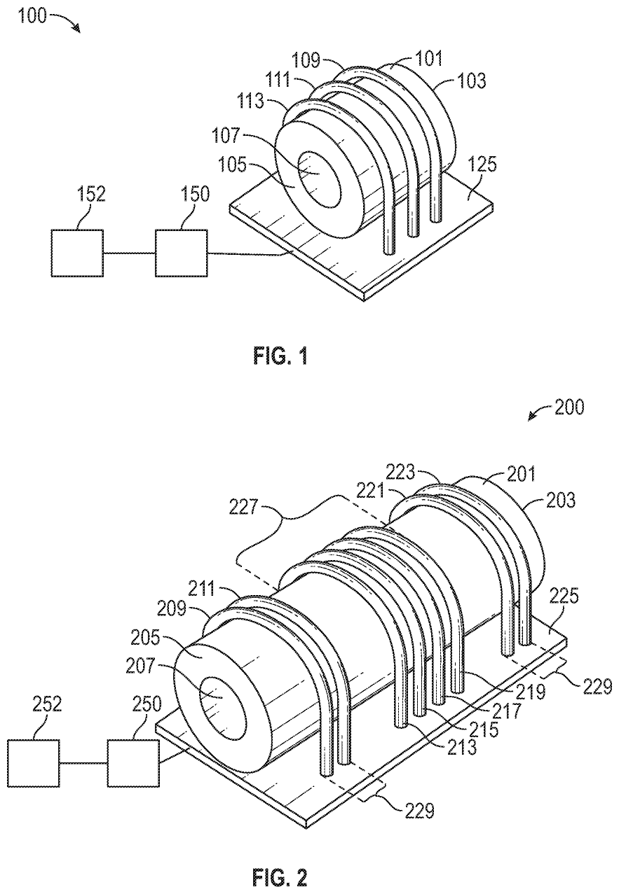

FIG. 1 illustrates a nanofluidic peristaltic pump 100 according to some embodiments of the present disclosure. The pump 100 includes an elongated tubular member 101 having a first end 103, an opposed second end 105, and a fluid flow channel 107 extending between the first end 103 and the second end 105. For example, the elongated tubular member may be elastomeric tubing. The pump 100 further includes a series of actuator wires 109, 111, 113, each comprising a shape memory alloy, and a substrate 125. The elongated tubular member 101 is secured between the substrate 125 and the actuator wires 109, 111, 113. The ends of the actuator wires are affixed to the substrate 125. The actuator wires 109, 111, 113 wrap around part of the circumferential surface of the tubular member 101, wrapping across the tubular member 101 in planes perpendicular to the longitudinal direction of the fluid flow channel 107. The tubular member 101 contacts the substrate 125 along one area of the outer surface of the tubular member 101 and contacts the actuator wires 109, 111, 113 along an opposed second area of the outer surface of the tubular member 101. The arrangement of the actuator wires, tubular member, and substrate is configured such that an electrothermally induced phase transition of the actuator wires compresses the tubular member in an amount effective to constrict the flow channel. The actuator wires 109, 111, 113 are further operably connected to a controller 150 and a power source 152 configured to deliver an electric current independently through each of the actuator wires 109, 111, 113. Although not shown, the substrate 125 may include electrical connectors for this purpose.

In use, the controller 150 and the power source 152 are operably connected and configured to selectively deliver an electric current to each of the actuator wires 109, 111, 113. Upon the application of electric current, each of the actuator wires 109, 111, 113 undergoes an electrothermally induced phase transition, causing each of the actuator wires 109, 111, 113 to reversibly and directly compress the elastic wall of elongated tubular member 101, and thereby constrict regions of the flow channel 107 between the first end 103 and the second end 105. The sequential constriction is effective to displace a fluid located within the fluid flow channel 107 in a peristaltic manner. In some embodiments, the region of deformation of an area of the elastic wall associated with deformation of one of the actuator wires does not overlap with that of neighboring actuator wires. The spacing between adjacent wires can be selected as needed to position regions of deformation next to or near one another in a non-overlapping fashion.

An electric current may be delivered to each of the actuator wires 109, 111, 113 in series. For example, an electric current may be delivered first to actuator wire 109, then actuator wire 111, and then actuator wire 113, to pump a fluid within the fluid flow channel 107 in a direction from the first end 103 toward the second end 105. In another embodiment, the electric current may be delivered first to actuator wire 113, then actuator wire 111, and then to actuator wire 109, to pump fluid within the fluid flow channel 107 in a direction from the second end 105 toward the first end 103.

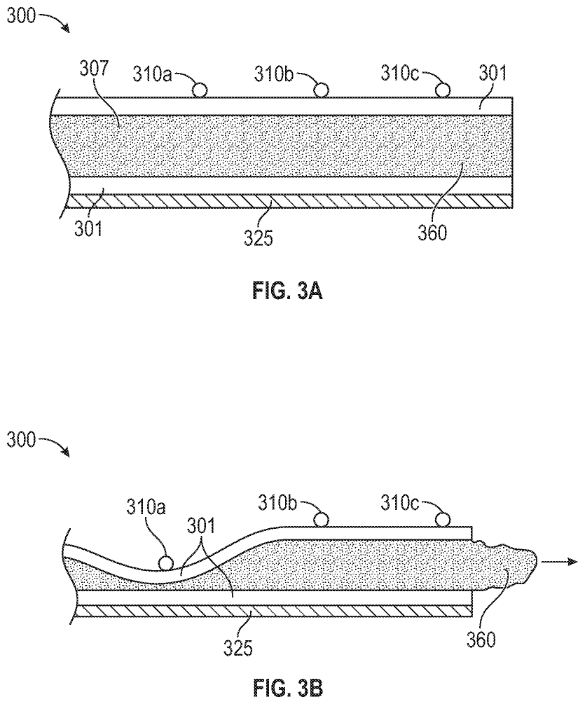

FIGS. 3A-3B illustrates an embodiment of the constriction-induced (peristaltic) flow of the presently disclosed nanofluidic peristaltic pumps, to show how the pumps operate. In FIG. 3A, nanofluidic peristaltic pump 300 includes elongated tubular member 301 positioned between, and in direct contact with, substrate 325 and actuator wires 310a, 310b, and 310c. The tubular member 301 includes fluid flow channel 307, which is filled with fluid 360. In FIG. 3A, none of the actuator wires 310a, 310b, and 310c are activated, and accordingly the fluid flow channel 307 is open and unconstricted. In FIG. 3B, however, actuator wire 310a is activated (e.g., receiving or having just received an electric current), and consequently contracted to constrict against the elastic elongated tubular member 301. This constriction elastically deforms a portion of the wall of the elastic elongated tubular member 301, causing it to collapse a portion of fluid flow channel 307 and thereby displacing the fluid 360 from flow channel 307, as shown.

The Elongated Tubular Member

The elongated tubular member may be constructed of any suitable material(s) that can be compressed and that are compatible with the fluid to be transported and the environment of use. In some embodiments, the elongated tubular member comprises an elastomeric material. In some embodiments, the elongated tubular member comprises a biocompatible elastomeric material. For example, in some embodiments, the elongated tubular member comprises silicone or polyurethane. In some embodiments, the tubular member is formed of a thermoplastic elastomer, such as styrene ethylene butylene styrene (SEBS).

In various embodiments, the tubular member is formed by a molding, casting, extrusion, or additive manufacturing process, adapted or known in the art. The flow channel may be formed simultaneously with the body of the tubular member. Alternatively, a subsequent process can be used in which a portion of the structural material is removed from the body in a region to define/form the flow channel.

In some embodiments, the elongated tubular member is constructed of a single material. In some other embodiments, the elongated tubular member is constructed of two or more materials, e.g., as a composite. The materials of construction may be biocompatible and suitable for sterilization, e.g., by gamma irradiation.

The elongated tubular member may be of any suitable dimensions that permit/provide peristaltic pumping. The elongated tubular member may have an annular shape. The cross-sectional shapes of the tubular member and the flow channel may be circular, or, alternatively, non-circular in some embodiments.

In some embodiments, the flow channel has a diameter of from about 20 μm to about 1000 μm. For example, the diameter may be from 50 μm to 500 μm, or from 100 μm to 500 μm. The diameter is one factor in selecting a suitable flowrate and liquid hold up volume for a particular application of the pump. The inner diameter of the flow channel may be directly proportional to the flow rate of the pump, such that reducing the inner diameter of the tubular member will reduce the single stroke volume, thereby allowing more precise nanofluidic control.

The wall thickness of the elongated tubular member may be selected to be mechanically robust, sufficiently flexible and collapsible, and remain fluid-tight over an extended period. For example, in some embodiments, the tubular member is constructed of a silicone and has a wall thickness ranging from 200 to 1000 microns.

Any material soft enough to elastically deform in response to the forces provided by the selected actuator wires may be used to construct the tubular member. For example, a nitinol wire may exert a pull force of 5.5 N when it contracts, so a suitable material of construction will deform in response to forces of this dimension.

Actuator Wires

The actuator wires may be dimensioned and constructed in essentially any manner that provides the required transformation to constrict the elongated tubular member of the pump. In a preferred embodiment, the actuator wires are formed of, or include, a shape memory alloy. In a preferred embodiment, the shape memory alloy is nickel titanium (nitinol). In some embodiments, the shape memory alloy is selected to be compatible with magnetic resonance imaging (MRI) so that the material is suitable for long term implantation in a patient.

In some embodiments, the actuator wires provide their reversible constriction function by undergoing a temperature-induced phase transition. For example, nitinol's high electrical resistance drives ohmic heating when current is passed through it, and this heating triggers a martensite to austenite phase transition in the alloy, which results in a physical contraction of a nitinol wire. Deactivating the electrical current cools the wire, causing the reverse phase transition and physical expansion of the wire. In this way, the physical contraction of the nitinol wire is used to reversibly and directly compress the elastic wall of the elongated tubular member. Other alloys and other materials may similarly use electrical resistance heating to drive contraction and expansion of the actuator wire.

Examples of other shape memory alloys that may be used in some embodiments include Ag—Cd 44/49 at. % Cd, Au—Cd 46.5/50 at. % Cd, Cu—Al—Ni 14/14.5 wt % Al and 3/4.5 wt % Ni, Cu—Sn approx. 15 at % Sn, Cu—Zn 38.5/41.5 wt. % Zn, Cu—Zn—X (X═Si, Al, Sn), Fe—Pt approx. 25 at. % Pt, Mn—Cu 5/35 at % Cu, Fe—Mn—Si, Co—Ni—Al, Co—Ni—Ga, Ni—Fe—Ga, Ti—Nb, Ni—Ti approx. 55-60 wt % Ni, Ni—Ti—Hf, Ni—Ti—Pd, and Ni—Mn—Ga.

In some embodiments, each of the actuator wires has a diameter of from about 25 μm to about 500 μm, e.g., from about 25 μm to about 100 am. For example, the wire diameter may be 50 μm, 60 μm, 70 μm, 80 μm, 90 μm, or within a range bound by a pair of these values. In some of these embodiments, the actuator wire is a nitinol wire.

The number of actuator wires and their spacing may depend on the various design parameters of the pump, including the length of the pump (i.e., the length of the flow channel) and the presence and number of check valves (described below), if any, to be included the pump.

In some embodiments, the series of actuator wires includes from 3 to 300 wires. In some embodiments, the pump has from 3 to 30 actuator wires. For example, the pump may include from 3 to 10 actuator wires, e.g., 3, 4, 5, 6, 7, 8, 9, or 10 actuator wires. Other numbers of wires are also envisioned depending on the particular application.

Pump with Check Valves

In some embodiments, the actuator wires, or at least a portion of a series of actuator wires, are configured to function as one or more check valves, to prevent back flow. For example, an actuator wire in an activated, or contracted, state may completely constrict the flow channel such that essentially no fluid can flow through the channel at that cross-sectional point in the channel.

FIG. 2 illustrates an embodiment of a nanofluidic peristaltic pump 200 that includes check valves. The pump 200 includes an elongated tubular member 201 having a first end 203, an opposed second end 205, and a fluid flow channel 207 extending between the first end 203 and the second end 205. The pump 200 further includes a series of actuator wires 209, 211, 213, 215, 217, 219, 221, 223, each of which may be formed of nitinol, and a substrate 225. The elongated tubular member 201 is secured between the substrate 225 and the actuator wires. The ends of the actuator wires are affixed to the substrate 225. The tubular member 201 contacts the substrate 225 along one area of the outer surface of the tubular member 201 and contacts the actuator wires along an opposed second area of the outer surface of the tubular member 201. The arrangement of the actuator wires, tubular member, and substrate is configured such that an electrothermally induced phase transition of the actuator wires compresses the tubular member in an amount effective to constrict the flow channel.

A first portion 227 of the actuator wires (actuator wires 213, 215, 217, 219) is configured to function as an actuator to pump fluid within the fluid flow channel 207, and second portions 229 of the actuator wires (actuator wires 209, 211, 221, 223) are configured to function as check valves to prevent back flow within the fluid flow channel 207. That is, wires 227 operate as actuators, and wires 229 operate as valves. The actuator wires are further operably connected to a controller 250 and a power source 252 configured to deliver an electric current independently through each of the actuator wires. Although not shown, the substrate 225 may include electrical connectors for this purpose.

In use, the controller 250 and the power source 252 are operably connected and configured to selectively deliver an electric current to each of the actuator wires. The timing of the activation and deactivation of the wires are coordinated to provide the flow and check valve functionality. For example, the actuator wires 213, 215, 217, and 219 may be activated sequentially to cause fluid flow within the fluid flow channel 207. For example, an electric current may be delivered first to actuator wire 213, then to actuator wire 215, then to actuator wire 217, and then to actuator wire 219, to cause fluid to flow within the fluid flow channel 207 from the second end 205 to the first end 203. An electrical current may be delivered to actuator wires 209 and 211 to prevent backflow within the fluid flow channel 207 from the first end 203 to the second end 205. To clarify, once the last actuator wire in a sequence relaxes, there can be some backflow, so the check valves are activated before the last actuator wire in a flow sequence relaxes, and remain activated until the next cycle of actuator wire activations and deactivations begins.

Alternatively, the actuator wires may be activated in reverse order to cause fluid to flow within the fluid flow channel 207 from the first end 203 to the second end 205. For example, an electric current may be delivered first to actuator wire 219, then to actuator wire 217, then to actuator wire 215, and then to actuator wire 213, to cause fluid to flow within the fluid flow channel 207 from the first end 203 to the second end 205. An electrical current may be delivered to actuator wires 221 and 223 to prevent backflow within the fluid flow channel from the second end 205 to the first end 203.

In some embodiments, the elongated tubular body is compressed between the actuator wire and a substrate, e.g., as in the illustrated embodiment, wherein the actuator wire is wrapped partly around the cross-section of the tubular body. In some other embodiments, the substrate is omitted and the elongated tubular body is compressed solely by the actuator wire, e.g., wherein the actuator wire is wrapped completely or nearly completely around the cross-section of the tubular body.

In some alternative embodiments, backflow is eliminated by incorporating a mechanical check valve in the tubing, rather than an electrical shape memory alloy wire-driven check valve. Such mechanical check valves are known in the art. Non-limiting examples include ball check valves, diaphragm check valves, and duckbill valves. In some other embodiments, the pump system may include a combination of one or more mechanical check valves and one or more shape memory alloy wire-driven check valves.

Other Components

In embodiments, the nanofluidic peristaltic pump includes means selectively delivering an electric current to each of the actuator wires, and in some embodiments, the means are configured to deliver electric current independently to each actuator wire.

The nanofluidic peristaltic pump includes a power source and a controller configured to selectively deliver an electric current, typically individually, to each of the actuator wires. The power source may be a battery or capacitor, for example. The controller may be a microcontroller, as known in the art.

In some embodiments, the substrate to which the ends of the actuator wires are fixed includes leads connected to the controller and the power source. In some embodiments, the substrate is a printed circuit board (PCB) and the controller and the power source are built into or upon the PCB.

In some other embodiments, the pump may be wirelessly powered and controlled, wherein the controller and the power source are remote from the pump. In one example, the nanofluidic peristaltic pump may be implanted subcutaneously in a patient, and the controller and/or power source are external to the patient, e.g., in a patch worn on the skin or scalp of the patient, with power and/or control signals wirelessly transmitted transcutaneously to the pump.

In some embodiments, the nanofluidic peristaltic pump includes a substrate on which the elongated tubular member is disposed and to which the actuator wires are affixed. In some embodiments, the power source and controller are also disposed on the substrate. For example, the power source and controller may be disposed on the same surface of the substrate as the elongated tubular member, or may be disposed on an opposite surface from the elongated tubular member. In some embodiments, the elongated tubular body, substrate, actuator wires, the power source, and the controller all are part of a medical implant device. In some other embodiments, the elongated tubular body and actuator wires are implantable in a patient while the power source and controller, and optionally the substrate, are external to the patient's body.

In some embodiments, the power source and controller allow for independent control of each of three or more actuator wires. That is, in some embodiments, electrical current may be independently provided to each of the three or more actuator wires, such that they may be activated and deactivated independently of one another.

Pump System

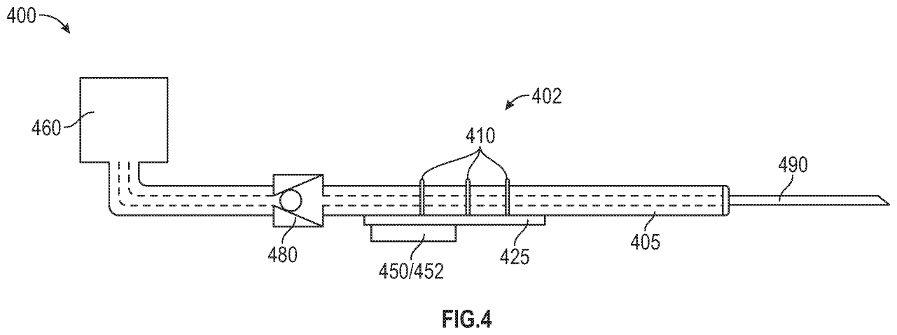

FIG. 4 illustrates one embodiment of a nanofluidic peristaltic pump system 400. The system 400 includes nanofluidic peristaltic pump 402 operably connected to and in fluid communication with a fluid source 460 and a microtube 490. The nanofluidic peristaltic pump 402 include elastomeric tubular member 405, actuator wires 410, and controller/power source 450/452. The actuator wires 410 and the controller/power source 450/452 are fixed to substrate 425. A mechanical check valve 480 is installed in-line with the nanofluidic peristaltic pump 402, preventing backflow of fluid toward the fluid source 460.

Other variations of this pump system (not shown) are envisioned. For example, the mechanical check valve may omitted and replaced with one or more actuator wire check valves. In another example, the microtube is omitted. In another example, a second mechanical check valve and/or an actuator wire check valve is/are included, for instance installed downstream of the nanofluidic peristaltic pump.

In yet another variation of the pump system, a multi-port valve (flow switch) is included between the nanofluidic peristaltic pump and the mechanical check valve. The multi-port valve may be particularly useful when the nanofluidic peristaltic pump 402 is a bidirectional pump. In such cases, the multi-port valve may have a first position for opening the fluid flow path between the fluid source and the microtube and closing off a sample receptacle, and a second position closing off the fluid source and opening the receptacle for fluids withdrawn from the site adjacent to the distal end of the microtube. Suitable multi-port valves are known in the art.

Operation of the Pump

Activation and deactivation of the actuator wires in an ordered manner drives the peristaltic motion of the elongated tubular body and thus the rate of fluid pumping therethrough.

Once all actuator wires are turned off following a directional flow cycle, relaxation of the wires and resultant relaxation of the tubing may result in backflow. Eliminating this backflow is critical to reliably and precisely controlling the movement of nanoliter fluid volumes. In some embodiments, elimination of backflow is accomplished by incorporating shape memory alloy wires into the pump that are configured to operate both as actuators to drive fluid flow and as valves to limit backflow, as illustrated for example in FIG. 2 described below. Once the actuator wires complete a directional flow cycle, the valve wires turn on to restrict fluid flow in the opposite direction following actuator wire relaxation. This mechanism can be used to precisely control bidirectional fluid flow with nanoliter precision in a robust and repeatable manner.

This may be illustrated with reference to the nanofluidic peristaltic pump shown in FIG. 2. First, the actuator wires 213, 215, 217, 219 are contracted in series to pump a fluid from the second end 205 to the first end 203 within the fluid flow channel 207. Specifically, the power source 252 and controller 250 are used to provide an electrical current to each of these actuator wires. By providing electrical current to these actuator wires, each wire is turned on (1) and off (0) as follows: 0000, 1000, 1100, 0100, 0110, 0010, 0011, 0001, 0000, where the first digit corresponds to the state of actuator wire 213, the second digit corresponds to the state of actuator wire 215, the third digit corresponds to the state of actuator wire 217, and the fourth digit corresponds to the state of actuator wire 219. Once all of the actuator wires 213, 215, 217, and 219 are turned off (0000), actuator wires 209 and 211 are activated to prevent backflow of fluid from the first end 203 to the second end 205 within the fluid flow channel 207. The actuator wires 209 and 211 are either turned on (1) and off (0) simultaneously (00, 11, 00) or in series (00, 10, 11, 00) to prevent backflow, where the first digit corresponds to the state of actuator wire 209 and the second digit corresponds to the state of actuator wire 211.

Since activation and deactivation of the actuator wires involves heating and cooling of the actuator wires, respectively, the heat and cool times for the actuator wires may determine the number of contraction cycles per minute and the resultant flow rate. Accordingly, it is believed that the flow rate of the nanofluidic peristaltic pump may be controlled by adjusting the heat and cool time of the actuator wires. For example, when using nitinol wires, it is believed that the heat and cool times can be reduced by reducing the diameter of the nitinol wire. For example, a 50 μm nitinol wire may be heated for only 1 second to accomplish contraction. Reducing wire diameter also reduces pull force exerted by the wire, prolonging the lifetime of the pump by reducing the fatigue experienced by the tubing.

In some embodiments, the nanofluidic peristaltic pump is configured to pump a fluid through the flow channel at a flow rate of 500 nL/s or less. For example, the flow rate may be from 1 nL/s to 500 nL/s. In various embodiments, the flow rate may be from 10 nL/s to 500 nL/s, from 20 nL/s to 450 nL/s, from 20 nL/s to 200 nL/s, from 50 nL/s to 400 nL/s, from 50 nL/s to 200 nL/s, from 50 nL/s to 150 nL/s, or from 30 nL/s to 300 nL/s.

In some embodiments, the nanofluidic peristaltic pump includes a first portion of the actuator wires in the series which are configured to be activated and deactivated sequentially to control bidirectional fluid flow through the flow channel. For example, in some embodiments the first portion of actuator wires in the series includes at least two actuator wires. Sequential activation and deactivation of these actuator wires in either sequence may be used to control bidirectional flow through the flow channel. For example, in some embodiments, the nanofluidic peristaltic pump may be used to collect a liquid sample from a patient, by activating the actuator wires in a first sequence to cause flow in a first direction through the fluid flow channel, and later may be used to deliver a drug to the patient, by activating the actuator wires in the opposite sequence to cause flow in the opposite direction through the fluid flow channel.

In some embodiments, the nanofluidic peristaltic pump includes a second portion of the actuator wires in the series which are configured to provide a check valve to prevent backflow in the flow channel. For example, in some embodiments the nanofluidic peristaltic pump includes a second portion of the actuator wires which are spaced apart from the first portion of the actuator wires and closer to the first end or the second end of the elongated tubular member than the first portion of the actuator wires. For example, in some embodiments the nanofluidic peristaltic pump includes at least one actuator wire closer to the first end or the second end of the elongated tubular member than the first portion of the actuator wires.

### System for Pumping Fluids

In some embodiments, the pump is part of pumping system configured for fluid delivery, for fluid withdrawal, or for both fluid delivery and withdrawal. In embodiments, the nanofluidic peristaltic pump described herein is coupled to a fluid source.

In some embodiments of a system with a bidirectional nanofluidic peristaltic pump as described herein, the system may include a multi-directional valves such that a fluid may be delivered in and withdrawn out of the same end of the pump, but into or from different fluid conduits. For example, the multi-directional valve may have a first position wherein the pump is in fluid communication with the fluid source and closed off from a collection vessel, and a second position wherein the pump is in fluid communication with the collection vessel and closed off from the fluid source.

### Uses/Applications of the Pump

The nanofluidic peristaltic pump described herein may be used in a wide variety of applications and industries, particularly where the transport of small quantities of fluid in precise volumes is needed.

Biomedical Applications

In some embodiments, the nanofluidic peristaltic pump is configured for biomedical applications, including but not limited to drug delivery and withdrawal of biological fluids for diagnostic analysis. The pump may be part of a portable or benchtop system configured for external, non-invasive fluid transport (e.g., in a handheld diagnostic device), or it may part of a system configured for in vivo fluid transport (e.g., biological fluid sampling and drug delivery).

For example, in some embodiments, the nanofluidic peristaltic pumps provided herein can be used to take liquid biopsies from the body of a patient, which may be used to identify disease type, state, and progression. In some embodiments, the nanofluidic peristaltic pumps can then be used to deliver drugs, of specific volumes and administration timelines, to targeted regions of the body. Unlike prior pumps, the nanofluidic peristaltic pumps provided herein are capable of bidirectional fluid flow, and in some embodiments, the nanofluidic peristaltic pumps provided herein are capable of more precise low-volume control than prior pumps.

In Vivo

In some embodiments, the step of providing the nanofluidic peristaltic pump includes implanting or inserting all or a portion of the nanofluidic peristaltic pump into the body of a patient. For example, in some embodiments, the entire nanofluidic peristaltic pump is implanted subcutaneously in the patient and is used to deliver a drug into the patient, to withdraw a sample of a biological fluid from the patient, or both. In some embodiments, the step of providing the nanofluidic peristaltic pump includes implanting or inserting only a portion of the nanofluidic peristaltic pump into the body of a patient. For example, in some embodiments the step of providing the nanofluidic peristaltic pump includes implanting the elongated tubular member and the actuator wires within a patient, while the controller and power source may remain outside the body of a patient.

The relatively slim-profile of the nanofluidic peristaltic pumps enables atraumatic design of embodiments which may facilitate subcutaneous implantation and interfacing with implanted medical devices at different tissue sites. Moreover, the small size of the pump may facilitate in vivo use of the pump over an extended period, e g., several days or months. This, in turn, enables minimally invasive sampling from and drug delivery to a range of tissues and organs, including tissues and organs where implantation of drug delivery devices was not previously possible, such as within the skull or the brain of a patient.

In some embodiments, a medical device insertable or implantable in a patient is provided, which includes a nanofluidic peristaltic pump as described herein. For example, the medical device may be configured for subcutaneous implantation in a patient for drug delivery and/or fluid sampling. In some embodiments the elongated tubular member and the actuator wires may be subcutaneously implanted and the power source and controller may be located outside of the patient's body.

Drug Delivery

In one example, the nanofluidic peristaltic pump is configured to transport a fluid comprising a drug, from a fluid source comprises the fluid to a delivery site distal from the fluid source. In such an embodiment, the flow channel at one end of the tubular member of the pump is in fluid communication with the fluid source and the opposed second end of the tubular member is in fluid communication with the delivery site. The sequential activation and deactivation of the actuator wires causes the drug-containing fluid to flow from the fluid source, through the flow channel from the first end toward the second end, and to the delivery site.

In one example, the nanofluidic peristaltic pump is part of a neural implant. In some embodiments, one or more microtubes are included between the second end (the discharge end) of the tubular member of the pump and the delivery site. That is, the microtubes are operably in fluid communication with the pump. Such microtubes serve as fluid conduits, or infusion channels. In some preferred embodiments, the microtube is an annular structure with an annulus size small enough to minimize/eliminate diffusion of the drug fluid when the system is in the off state, thereby enabling pinpoint, sub-mm³ volume dosing. For example, in one embodiment, the microtube has an outer diameter of about 30 microns and an inner diameter of about 20 microns. The microtube may be formed of any suitable material, such as a biocompatible material that is also compatible with the drug fluid. In some preferred embodiments, the microtube is formed of a borosilicate glass.

In some embodiments, the fluid includes the drug and a liquid excipient vehicle for the drug. For example, in some embodiments, the fluid includes a drug and water or a saline solution. Other suitable excipients are known in the art and may be included as appropriate. The drug may be essentially any prophylactic or therapeutic agents, or any active pharmaceutical ingredient, known in the art. The fluid drug may include a neuromodulating agent. In some embodiments, the neuromodulating agent comprises muscimol or another GABA agonist. Other neuromodulating agents known in the art also may be used.

Fluid Withdrawal

In one example, the nanofluidic peristaltic pump is configured to withdraw a biological fluid from a site in vivo. In such an embodiment, the flow channel at a distal end of the tubular member of the pump is in fluid communication with the site of the fluid to be withdrawn or sampled, and the opposed proximal end of the tubular member is in fluid communication with a collection vessel and/or sensor. The sequential activation and deactivation of the actuator wires causes the biological fluid to flow from the in vivo site, through the flow channel from the distal end toward the proximal end, and to the collection vessel and/or diagnostic sensor.

In some embodiments, the biological fluid is blood, cerebrospinal fluid, or interstitial fluid. Other biological fluids are also envisioned.

In some embodiments, the sensor is a diagnostic sensor configured to detect various analyte levels or pH. The sensor may also detect or measure other properties of the biological fluid.

In some embodiments, a method of use includes first providing a nanofluidic peristaltic pump as described above, with the flow channel at the first end of the tubular member in fluid communication with a biological and the second end in fluid communication with one or more sensors and a drug source; and delivering an electric current to at least the first portion of the actuator wires to sequentially activate and deactivate them and cause the biological fluid to flow through the flow channel from the first end toward the second end toward one or more sensors configured to detect a characteristic or component of the biological fluid. Next, depending on the characteristic of or component (e.g., a particular analyte of interest) in the biological fluid, the method further includes delivering an electric current to at least the first portion of the actuator wires to sequentially activate and deactivate them and cause the drug to flow from the drug source through the flow channel from the second end to the first end toward the body of a patient.

In some embodiments, the method further includes delivering an electric current to at least a second portion of the actuator wires to activate them as a check valve to prevent backflow of the fluid in the flow channel toward the fluid source. For example, in some embodiments the method further includes first delivering an electric current to the first portion of the actuator wires to initiate fluid flow and, once electric current is no longer delivered to the first portion of the actuator wires, delivering an electric current to the second portion of the actuator wires to activate them as a check valve to prevent backflow of the fluid in the flow channel toward the fluid source.

Non-Medical Applications

In other embodiments, the nanofluidic peristaltic pumps described herein may be used to interface with other devices, e.g., other microfluidic or nanofluidic devices. For example, the pump may be used to provide cooling fluid to electronic devices and electrical components, driving fluid flow within these devices with high precision while retaining its compact design and small physical footprint. The cooling fluid may be aqueous, for example.

The devices and methods described herein will be further understood by reference to the following non-limiting examples.

### Example 1: A Nanofluidic Peristaltic Pump

A nanofluidic peristaltic pump was prepared and tested to determine stroke volume and flow rate. A tubing having an outer diameter of 1 mm and an inner diameter of 500 am forming a fluid flow channel between a first end and a second end of the tubing was used to create a nanofluidic peristaltic pump. The tubing material of construction was styrene ethylene butylene styrene (SEBS). The tubing was placed on a substrate, and a 100 am nitinol wire was affixed to the substrate and in contact with the outer surface of the tubing. The substrate material of construction was acrylonitrile butadiene styrene (ABS). Nitinol wire was threaded through holes in the substrate. The ends of the nitinol wire were clamped with crimp beads, which were soldered to a circuit on a breadboard. The circuit was powered by a benchtop power source and controlled by an Arduino. The current applied was 180 mA per wire. Water, containing a dye for ease of flow visualization, was used as the fluid to be pumped.

An electric current was provided to the nitinol wire for two seconds to cause contraction of the nitinol wire, and compression of the wall of the tubing and flow channel. The flow rate was calculated by taking a video of the fluid moving inside the clear tubing using a microscope. Using video analysis software, the fluid meniscus was tracked over time, and knowing the dimensions of the fluidic channel, the resultant flow rate was calculated. The amount of fluid pumped during this time was measured to be 196 nL. Since the nitinol wire was heated for 2 seconds to accomplish this fluid flow, the flow rate was calculated to be 98 nL/s.

In summary, the contraction of a single wire was demonstrated on compliant tubing with an inner diameter of 500 μm generates a single stroke volume of 196 nL, with a resultant flow rate of 98 nL/s.

### Example 2: A Second Nanofluidic Peristaltic Pump

Tube inner diameter is directly proportional to flow rate. Reducing the tube inner diameter will reduce the single stroke volume, allowing precise nanofluidic control.

Another nanofluidic peristaltic pump was prepared and tested to determine stroke volume and flow rate, like in Example 1, except with a tubing inner diameter of 100 m. The flow rate was reduced to 65 nL/min.

### Example 3: A Third Nanofluidic Peristaltic Pump

The heat and cool times for the nitinol wires, which determine the number of contraction cycles per minute and the resultant flow rate, can be decreased by reducing the diameter of the wire.

### Example 4: A Fourth Nanofluidic Peristaltic Pump

In another example, combining insights from Examples 2 and 3, the tubing inner diameter was reduce to 100 microns, which reduced the stroke volume resulting from a single wire contraction. A 50 μm diameter nitinol wire was used; contraction of the wire could be accomplished by heating for 1 second. We also tuned the flow control algorithm to (1) reduce the amount of time the wire is heated (causing it to contract less) and (2) alter the overlap time between two adjacent wires contracting (determining the efficiency of directional flow). A flow rate of 1 nL/s was achieved.

### Exemplary Embodiments

### Embodiment 1

A nanofluidic peristaltic pump comprising: an elongated tubular member having a first end, an opposed second end, and a wall defining a flow channel extending between the first and second ends; and a series of actuator wires, each comprising a shape memory alloy, wherein the actuator wires extend across and at least partially around the outer surface of the elastic wall at spaced positions along the length of the tubular member, the actuator wires being configured to reversibly and directly compress the wall, and thereby constrict regions of the flow channel, upon an electrothermally induced phase transition of the shape memory alloy.

### Embodiment 2

The nanofluidic peristaltic pump of embodiment 1, further comprising a power source and a controller configured to selectively deliver an electric current to each of the actuator wires.

### Embodiment 3

The nanofluidic peristaltic pump of embodiment 1 or 2, wherein at least a first portion of the actuator wires in the series are configured to be activated and deactivated sequentially to control bidirectional fluid flow through the flow channel.

### Embodiment 4

The nanofluidic peristaltic pump of any one of embodiments 1 to 3, wherein at least a second portion of the actuator wires in the series are configured to provide a check valve to prevent backflow in the flow channel.

### Embodiment 5

The nanofluidic peristaltic pump of any one of embodiments 1 to 4, wherein the shape memory alloy comprises or consists of nitinol.

### Embodiment 6

The nanofluidic peristaltic pump of any one of embodiments 1 to 5, wherein the elongated tubular member comprises silicone, polyurethane, or styrene ethylene butylene styrene.

### Embodiment 7

The nanofluidic peristaltic pump of any one of embodiments 1 to 6, wherein the series of actuator wires comprises from 3 to 300 wires.

### Embodiment 8

The nanofluidic peristaltic pump of any one of embodiments 1 to 7, further comprising a substrate on which the elongated tubular member is disposed and to which the actuator wires are affixed.

### Embodiment 9

The nanofluidic peristaltic pump of any one of embodiments 1 to 8, wherein each of the actuator wires has a diameter from about 50 μm to about 100 μm.

### Embodiment 10

The nanofluidic peristaltic pump of any one of embodiments 1 to 9, wherein the flow channel has a diameter from about 20 μm to about 1000 μm.

### Embodiment 11

The nanofluidic peristaltic pump of any one of embodiments 1 to 10, which is configured to pump a fluid through the flow channel at a flow rate of 500 nL/s or less, for example between 1 nL/s and 500 nL/s, for example, 50 nL/s and 100 nL/s.

### Embodiment 12

The nanofluidic peristaltic pump of any one of embodiments 1 to 11, which is configured to pump a fluid through the flow channel at a flow rate of about 100 nL/s.

### Embodiment 13

The nanofluidic peristaltic pump of any one of embodiments 1 to 12, further comprising one or more mechanical check valves in fluid communication with the flow channel to prevent backflow in the flow channel.

### Embodiment 14

A medical device comprising: the nanofluidic peristaltic pump of any one of embodiments 1 to 13, wherein the nanofluidic peristaltic pump is configured to be insertable or implantable in a patient.

### Embodiment 15

The medical device of any one of embodiments 1 to 14, which is configured for subcutaneous implantation in a patient for drug delivery and/or fluid sampling.

### Embodiment 16

A method of pumping a fluid, the method comprising: providing the nanofluidic peristaltic pump of any one of embodiments 1 to 15 with the flow channel at the first end of the tubular member in fluid communication with a fluid source; and delivering an electric current to at least first portion of the actuator wires to sequentially activate and deactivate them and cause the fluid to flow through the flow channel from the first end toward the second end.

### Embodiment 17

The method of embodiment 16, further comprising delivering an electric current to at least a second portion of the actuator wires to activate them as a check valve to prevent backflow of the fluid in the flow channel toward the fluid source.

### Embodiment 18

The method of embodiment 16 or 17, wherein the fluid comprises a biological fluid.

### Embodiment 19

The method of embodiment 16 or 17, wherein the fluid comprises a drug and a liquid excipient vehicle for the drug.

### Embodiment 20

The method of any one of embodiments 16 to 19, wherein the step of providing the nanofluidic peristaltic pump comprises implanting or inserting the nanofluidic peristaltic pump into the body of a patient.

### Embodiment 21

The method of embodiment 20, wherein the nanofluidic peristaltic pump is implanted subcutaneously in the patient and is used to deliver a drug into the patient, to withdraw a sample of a biological fluid from the patient, or both.

### Embodiment 22

A bidirectional nanofluidic peristaltic pump comprising: an elongated, elastomeric tubular member having a first end, an opposed second end, and a wall defining a flow channel extending between the first and second ends; and a series of nitinol actuator wires extending around at least part of the outer surface of the wall of the elastomeric tubular member, the nitinol actuator wires being in contact with the wall and at positions spaced from one another; and a power source and controller operably connected to the series of actuator wires and configured to selectively sequentially deliver an electric current to each of the nitinol actuator wires to electrothermally induce a phase transition of the nitinol, wherein the actuator wires, upon the electrothermally induced phase transition of the nitinol, are configured to reversibly and directly compress the wall, and thereby constrict regions of the flow channel.

### Embodiment 24

The bidirectional nanofluidic peristaltic pump of embodiment 23, wherein the elastomeric tubular member comprises silicone, polyurethane, or styrene ethylene butylene styrene.

### Embodiment 25

The bidirectional nanofluidic peristaltic pump of embodiment 23 or 24, wherein the series of actuator wires comprises from 3 to 300 wires.

### Embodiment 26

The bidirectional nanofluidic peristaltic pump of any one of embodiments 23 to 25, further comprising a substrate on which the elastomeric tubular member is disposed and to which the nitinol actuator wires are affixed.

### Embodiment 27

The bidirectional nanofluidic peristaltic pump of any one of embodiments 23 to 26, wherein each of the actuator wires has a diameter from about 50 μm to about 100 μm and the flow channel has a diameter from about 20 μm to about 1000 μm. Publications cited herein and the materials for which they are cited are specifically incorporated by reference. Modifications and variations of the methods and devices described herein will be obvious to those skilled in the art from the foregoing detailed description. Such modifications and variations are intended to come within the scope of the appended claims.

We claim:

1. A nanofluidic peristaltic pump comprising:
an elongated tubular member having a first end, an opposed second end, and an elastic wall defining a flow channel extending between the first and second ends; and
a series of actuator wires, each comprising a shape memory alloy,
wherein the actuator wires extend across and at least partially around the outer surface of the elastic wall at spaced positions along the length of the tubular member, the actuator wires being configured to reversibly and directly compress the wall, and thereby constrict regions of the flow channel, upon an electrothermally induced phase transition of the shape memory alloy.

2. The nanofluidic peristaltic pump of claim 1, further comprising a power source and a controller configured to selectively deliver an electric current to each of the actuator wires.

3. The nanofluidic peristaltic pump of claim 1, wherein at least a first portion of the actuator wires in the series are configured to be activated and deactivated sequentially to control bidirectional fluid flow through the flow channel.

4. The nanofluidic peristaltic pump of claim 3, wherein at least a second portion of the actuator wires in the series are configured to provide a check valve to prevent backflow in the flow channel.

5. The nanofluidic peristaltic pump of claim 1, wherein the shape memory alloy comprises nitinol.

6. The nanofluidic peristaltic pump of claim 1, wherein the elongated tubular member comprises silicone, polyurethane, or styrene ethylene butylene styrene.

7. The nanofluidic peristaltic pump of claim 1, wherein the series of actuator wires comprises from 3 to 300 wires.

8. The nanofluidic peristaltic pump of claim 1, further comprising a substrate on which the elongated tubular member is disposed and to which the actuator wires are affixed.

9. The nanofluidic peristaltic pump of claim 1, wherein each of the actuator wires has a diameter from about 50 μm to about 100 μm.

10. The nanofluidic peristaltic pump of claim 1, wherein the flow channel has a diameter from about 20 μm to about 1000 μm.

11. The nanofluidic peristaltic pump of claim 1, which is configured to pump a fluid through the flow channel at a flow rate of 500 nL/s or less.

12. The nanofluidic peristaltic pump of claim 11, which is configured to pump a fluid through the flow channel at a flow rate of about 100 nL/s.

13. The nanofluidic peristaltic pump of claim 11, which is configured to pump a fluid through the flow channel at a flow rate of between 50 nL/s and 100 nL/s.

14. The nanofluidic peristaltic pump of claim 1, further comprising a mechanical check valve in fluid communication with the flow channel to prevent backflow in the flow channel.

15. A medical device comprising: the nanofluidic peristaltic pump of claim 1, wherein the nanofluidic peristaltic pump is configured to be insertable or implantable in a patient.

16. The medical device of claim 15, which is configured for subcutaneous implantation in a patient for drug delivery and/or fluid sampling.

17. A method of pumping a fluid, the method comprising:
providing the nanofluidic peristaltic pump of claim 1 with the flow channel at the first end of the tubular member in fluid communication with a fluid source; and
delivering an electric current to at least first portion of the actuator wires to sequentially activate and deactivate them and cause the fluid to flow through the flow channel from the first end toward the second end.

18. The method of claim 17, further comprising delivering an electric current to at least a second portion of the actuator wires to activate them as a check valve to prevent backflow of the fluid in the flow channel toward the fluid source.

19. The method of claim 17, wherein the fluid comprises a biological fluid.

20. The method of claim 17, wherein the fluid comprises a drug and a liquid excipient vehicle for the drug.

21. The method of claim 17, wherein the step of providing the nanofluidic peristaltic pump comprises implanting or inserting the nanofluidic peristaltic pump into the body of a patient.

22. The method of claim 21, wherein the nanofluidic peristaltic pump is implanted subcutaneously in the patient and is used to deliver a drug into the patient, to withdraw a sample of a biological fluid from the patient, or both.

23. A bidirectional nanofluidic peristaltic pump comprising:
an elongated, elastomeric tubular member having a first end, an opposed second end, and a wall defining a flow channel extending between the first and second ends;
a series of shape memory alloy (SMA) actuator wires extending around at least part of the outer surface of the wall of the elastomeric tubular member, the SMA actuator wires being in contact with the wall and at positions spaced from one another; and
a power source and controller operably connected to the series of actuator wires and configured to selectively sequentially deliver an electric current to each of the SMA actuator wires to electrothermally induce a phase transition of the SMA,
wherein the SMA actuator wires, upon the electrothermally induced phase transition of the SMA, are configured to reversibly and directly compress the wall, and thereby constrict regions of the flow channel.

24. The bidirectional nanofluidic peristaltic pump of claim 23, wherein the elastomeric tubular member comprises silicone, polyurethane, or styrene ethylene butylene styrene.

25. The bidirectional nanofluidic peristaltic pump of claim 23, wherein the series of actuator wires comprises from 3 to 300 wires.

26. The bidirectional nanofluidic peristaltic pump of claim 23, further comprising a substrate on which the elastomeric tubular member is disposed and to which the actuator wires are affixed.

27. The bidirectional nanofluidic peristaltic pump of claim 23, wherein each of the SMA actuator wires has a diameter from about 50 μm to about 100 μm and the flow channel has a diameter from about 20 μm to about 1000 μm.

28. The bidirectional nanofluidic peristaltic pump of claim 23, wherein the SMA actuator wires comprise nitinol.
